(19) United States
(12) Patent Application Publication
(10) Pub. No.: US 2010/0326436 A1
(43) Pub. Date: Dec. 30, 2010

(54) INHALER

(75) Inventor: Hideki Kaneko, Yokohama-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/920,836

(22) PCT Filed: Apr. 30, 2009
(86) PCT No.: PCT/JP2009/058800

§ 371 Date: Sep. 3, 2010

(30) Foreign Application Priority Data
May 7, 2008 (JP) 2008-121102

(51) Int. Cl. A61M 15/00

(52) U.S. Cl. 128/203.12

(57) ABSTRACT

There is provided an inhaler capable of allowing a medicine to effectively reach a target site even when a plurality of types of medicines having different target sites is used in the same inhaler. In a control unit (CPU) of a housing, a medicine identification portion identifies the type of medicine stored in a medicine storing portion of a cartridge. A determination portion determines an air inhalation volume when the medicine is inhaled based on the type of medicine identified by the medicine identification portion. A medicine ejection portion is arranged in an air flow path. The medicine ejected from the medicine ejection portion is inhaled by a user from a suction port. A display portion of the housing informs the user of the inhalation volume determined by the determination portion.

### TECHNICAL FIELD

The present invention relates to an inhaler which can be brought along with a user to allow the user to inhale a medicine.

### BACKGROUND ART

Inhalers which allow a user to inhale a medicine have been developed. The inhalers allow inhalation by ejecting fine droplets of medicine into an air flow path where air inhaled through a mouthpiece flows by using an ejection principle of an inkjet system (see Japanese Patent Application Laid-Open No. 2004-290593 and Japanese Patent Application Laid-Open No. 2004-283245). Such inhalers have an advantage that a predetermined amount of medicine having a uniform particle diameter can be precisely ejected.

Other typical inhalers used for medical purposes include suspension aerosol type metered dose inhalers (MDI), dry powder inhalers (DPI), and nebulizers.

When treatment is provided using the inhaler as described above, it is necessary to allow a medicine to effectively reach its target site. For example, a target site in treating diabetes is a lung alveoli region where a medicine is easily absorbed from the capillaries. Thus, it is necessary to allow insulin to effectively reach the lung alveoli region. The insulin may be deposited on the oropharynx or bronchial region before reaching the lung alveoli. This is not preferable since the absorption rate of insulin into blood becomes slower, and the insulin might remain in the body. To obtain an adequate treatment effect, the amount of ejected insulin may be increased to increase the amount of insulin to reach the lung alveoli. However, increasing the amount of ejected insulin causes an increase in cost. Meanwhile, in treating bronchitis, a bronchodilator for dilating bronchi, such as salbutamol, targets a bronchial region. Thus, it is necessary to allow the bronchodilator to effectively reach the bronchi. The bronchodilator may reach the lung alveoli without being deposited on the bronchial region. This is not preferable since the bronchodilator is absorbed into blood from the capillaries, and the bronchi cannot be effectively treated. To obtain an adequate treatment effect, the amount of ejected bronchodilator may be increased to increase the amount of bronchodilator to reach the bronchi. However, increasing the amount of ejected bronchodilator causes an increase in cost.

To allow a medicine to effectively reach a target site, more attention has been paid on appropriate selection of not only the mass median aerodynamic diameter of medicine, which is conventionally known, but also the amount of inhaled air when the medicine is inhaled according to which site the medicine is to reach (see HIROSHI TAKANO, “PHARM TECH JAPAN” Vol. 20, No. 9, p. 165-173, 2004). To allow the medicine such as the insulin to effectively reach the lung alveoli, it is desirable for a user to inhale as much air as possible at the time of inhaling the medicine (see Japanese Patent Application Laid-Open No. 2002-504833). Also, to allow the medicine such as the bronchodilator to effectively reach the bronchial region, an inhalation volume approximate to that obtained during breathing at rest is desirable.

A plurality of types of medicines whose target sites are different from each other may be used in the same inhaler. In this case, it is difficult for a user himself or herself to inhale an appropriate amount of medicine for each medicine. For example, a patient who suffers from diabetes, and also suffers from asthma or bronchitis may take the bronchodilator and the insulin from the same inhaler. The bronchodilator targets the bronchial region, and the insulin targets the lung alveoli region. In this case, it is difficult for the user himself or herself to control an appropriate inhalation volume of air for each target site when inhaling each medicine.

If the user cannot inhale the appropriate inhalation volume of air for the target site of the medicine, the medicine cannot effectively reach the target site. In this case, the medicine is deposited on a position other than the target site, and effective treatment cannot be provided. Also, the amount of medicine to reach the target site may be increased to obtain an adequate treatment effect. In this case, the amount of ejected medicine is increased, which causes an increase in cost.

### DISCLOSURE OF THE INVENTION

It is an object of the present invention to provide an inhaler capable of selecting an appropriate inhalation volume for each medicine even when a plurality of types of medicines is used.

In view of the above object, the inhaler according to the present invention is characterized by including: a medicine storing portion for storing a medicine; a medicine ejection portion for ejecting the medicine; a suction port for allowing a user to inhale air including the ejected medicine; a medicine identification unit for identifying a type of medicine stored in the medicine storing portion; and a determination unit for determining an inhalation volume to be inhaled by a user according to the type of medicine identified by the medicine identification unit.

According to the inhaler of the present invention, the user can inhale the inhalation volume determined for each medicine by the determination unit. Accordingly, the medicine can effectively reach a target site.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIGS. 1A and 1B illustrate a basic configuration of an inhaler according to the present invention; FIG. 1A is a schematic view illustrating the entire inhaler; FIG. 1B is a view illustrating an internal configuration of a cartridge.

FIG. 2 is a schematic view illustrating a first embodiment.

FIG. 3 is a schematic view illustrating a second embodiment.

FIG. 4 is a schematic view illustrating a third embodiment.

FIGS. 5A, 5B and 5C illustrate a content displayed on a display portion in FIG. 4; FIG. 5A illustrates an informing content to inform a user of an appropriate inhalation volume; FIG. 5B illustrates an informing content to inform a relation between an inhalation volume of a user and an appropriate inhalation volume; FIG. 5C illustrates an informing content to provide information about an inhalation volume to a user during inhalation.

FIG. 6 is a schematic view illustrating a fourth embodiment.

FIG. 7 is schematic view illustrating an inhaler according to one example.

FIG. 8, which is composed of FIGS. 8A and 8B, is a flowchart illustrating a usage example of the inhaler in FIG. 7.

### BEST MODES FOR CARRYING OUT THE INVENTION

Preferred embodiments of the present invention will now be described in detail in accordance with the accompanying drawings.

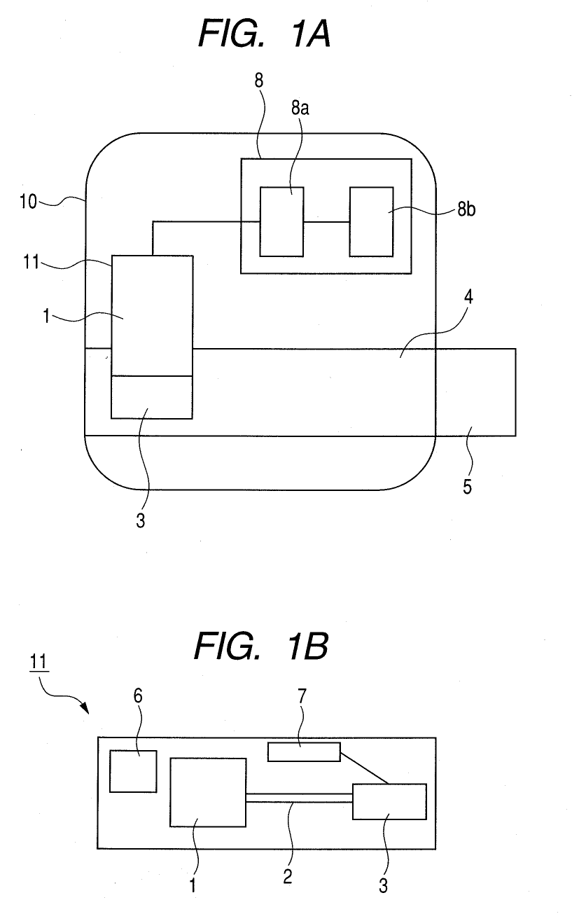

FIG. 1A illustrates a basic configuration of an inhaler according to the present invention. A cartridge 11 is detachably attached to a housing 10. The cartridge 11 is formed such that a medicine storing portion 1, a medicine flow path 2, and a medicine ejection portion 3 are integrated. The housing 10 includes an air flow path 4 and a suction port 5 which constitute a suction portion from which a user inhales air including a medicine. An authentication code 6 for allowing identification of the type of medicine and an electrical connection portion 7 are arranged in the cartridge 11. The housing 10 includes a control unit (CPU) 8 having a medicine identification portion 8a as a medicine identification unit. The medicine identification portion 8a identifies and selects the type of medicine stored in the medicine storing portion 1 of the cartridge 11. The control unit (CPU) 8 further includes a determination portion 8b as a determination unit. The determination portion 8b determines an inhalation volume of air to be inhaled by a user according to the type of medicine identified by the medicine identification portion 8a. The medicine ejection portion 3 is disposed in contact with the air flow path 4. A user inhales from the suction port 5 a medicine ejected from the medicine ejection portion 3 based on the inhalation volume determined by the determination portion 8b.

FIG. 1B illustrates an internal configuration of the cartridge 11. The medicine ejection portion 3, the medicine storing portion 1, and the medicine flow path 2 are integrally arranged on the same substrate. The medicine ejection portion 3 ejects a medicine. The medicine flow path 2 guides a medicine from the medicine storing portion 1 to the medicine ejection portion 3. A controller (a drive control portion) for controlling the drive of the medicine ejection portion 3 is provided in the housing 10. The controller and the medicine ejection portion 3 exchange drive signals and control signals via the electrical connection portion 7. The electrical connection portion 7 is connected by inside wiring thereto.

The cartridge 11 has the authentication code 6 for allowing identification of the type of medicine stored in the medicine storing portion 1. A well-known authentication unit distinguishable according to the type of medicine may be used as the authentication code 6 of the cartridge. The well-known authentication unit includes bar-codes, QR codes, RFID, and IC chips. A well-known method may be employed as a method of reading the authentication code. The well-known method includes methods of identification using images, electricity and electric waves. Specific examples thereof include CCDs, CMOSs, electrical contacts and antennas. The authentication code 6 is selected therefrom. Information regarding the type of medicine is recorded in the authentication code 6. However, the inhaler has only to identify the type of medicine. Thus, an embodiment in which the authentication code 6 is not provided in the cartridge 11 and a user inputs the type of medicine to the inhaler may be also employed, for example.

A plurality of cartridges may be also mounted on the inhaler. If cartridges storing a plurality of medicines respectively having different target sites are mounted on the inhaler, it is desirable to separately inhale the medicines since an optimum inhalation volume differs in each medicine.

According to the target site of the identified medicine, the inhaler of the present invention can determine an appropriate inhalation volume of air for the target site. When medicines to be delivered to a plurality of target sites are used, there is an appropriate air inhalation volume for each target site. As described above, to allow a medicine such as insulin to effectively reach lung alveoli, it is desirable for a user to inhale as much air as possible when inhaling the medicine. To allow a medicine such as a bronchodilator to effectively reach a bronchial region, an inhalation volume approximate to that obtained during breathing at rest is desirable. The inhaler realizes such a desirable inhalation volume. To realize such a desirable inhalation volume, information regarding the desirable inhalation volumes corresponding to the types of medicines used in the inhaler is stored in the determination portion 8b. The inhaler thereby identifies the type of medicine, and determines the appropriate air inhalation volume for the target site.

In the present invention, the “inhalation volume” means the amount of air which a user inhales in one inhalation. The inhalation volume is a different concept from an “ejection volume” which is the amount of ejected medicine, and a “dosage” which is the amount of medicine actually inhaled by a user.

A preferable air inhalation volume for allowing the medicine such as insulin to effectively reach lung alveoli is a volume close to a vital capacity (about 3000 mL for an adult male). The vital capacity is the maximum volume of air that a user can inhale. However, a user cannot always inhale the volume of air equivalent to the vital capacity. Thus, the optimum air inhalation volume for the medicine such as insulin can employ a volume smaller than the vital capacity of a user. To be more specific, the optimum air inhalation volume can be a volume of 60 to 99% of the vital capacity of a user.

At the time of carrying out the present invention, various proteins and peptides can be used as the medicine which targets the lung alveoli. Examples of the proteins and peptides include various hematopoietic factors such as calcitonin, blood coagulation factors, cyclosporine, G-CSF, GM-CSF, SCF, EPO, GM-MSF, and CSF-1. The examples also include interleukins, IGFs, M-CSF, thymosin, and cytokines. The examples further include interferons and hormones. The hormones include human growth hormones and growth hormones of other animals (such as bovine, porcine, and chicken growth factors).

To allow the medicine to reach the lung alveoli, the mass median aerodynamic diameter (MMAD) of medicine ejected from the medicine ejection portion can be 1 to 4 μm.

Meanwhile, to allow the medicine such as a bronchodilator to effectively reach a bronchial region, the inhalation volume obtained during breathing at rest is desirable. A user can reproducibly inhale the inhalation volume obtained during breathing at rest (about 500 mL for an adult male) without paying extra attention.

At the time of carrying out the present invention, the medicine which targets the bronchial region includes compounds as represented by antitussives, respiratory stimulants, bronchodilators, gargles, and expectorants, which are used for treating various organs with respiratory diseases such as asthma and chronic obstructive pulmonary disease (COPD). Specific examples of the active ingredients include cromoglycic acid, salbutamol, ipratropium, fenoterol, isoproterenol, trimetoquinol, procaterol, salmeterol, and oxitropium. The specific examples also include beclometasone dipropionate, bromhexine, acetylcysteine, budesonide, and fluticasone propionate. Partially-substituted derivatives of the compounds can be similarly employed.

To allow the medicine to reach the bronchial region, the mass median aerodynamic diameter (MMAD) of medicine ejected from the medicine ejection portion can be 5 to 10 μm.

The optimum inhalation volume may be also determined by checking information about the identified medicine with the vital capacity and the inhalation volume at rest of a user stored in a memory portion (ROM) in advance. In this manner, a difference in the vital capacity and the inhalation volume at rest of each user due to sex, age, and physical constitution can be taken into account.

In the present invention, the medicine ejection portion (an ejection head) includes any ejection pressure generating element. That is, the medicine ejection portion employs an ejection principle including powder ejection, an MDI system, a jet type nebulizer, an ultrasonic type nebulizer, a mesh type nebulizer, a cam push-out system, and an inkjet system, although not limited thereto. The ejection pressure generating element can employ an electrothermal transducer for applying thermal energy to a medicine, and an electromechanical transducer for applying mechanical energy to a medicine. That is, an ejection method includes a method of ejecting a medicine from an ejection port by applying thermal energy to the medicine by using the electrothermal transducer (a thermal jet system). The ejection method also includes a method of ejecting a medicine from an ejection port by using a vibratory pressure of the electromechanical transducer (for example, a piezoelectric element) which applies mechanical energy to the medicine. The ejection method may be selected according to the type of medicine.

The inkjet system such as the thermal jet system has an advantage that a predetermined amount of medicine having a uniform particle diameter can be precisely ejected.

### First Embodiment

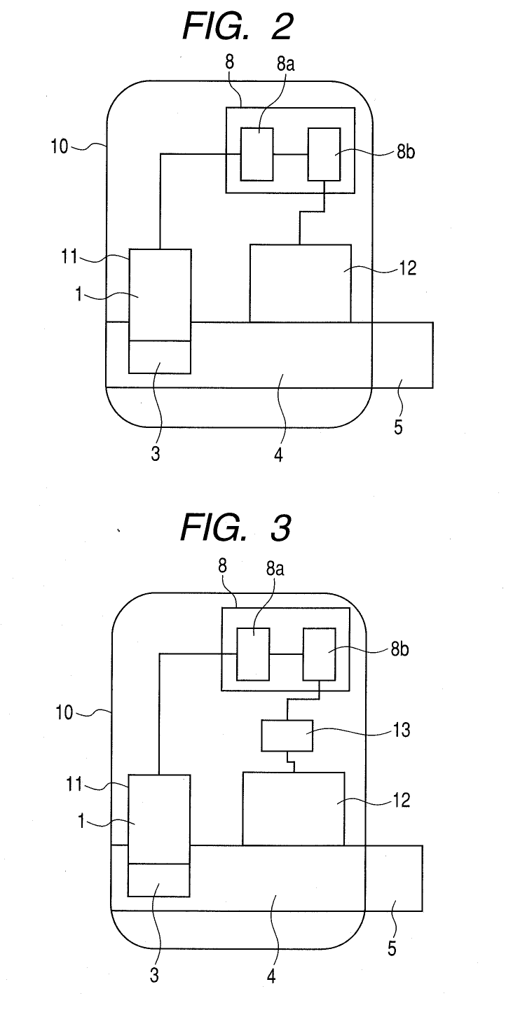

FIG. 2 illustrates a first embodiment. Here, an inhalation volume sensor 12 is attached to the apparatus in FIG. 1A. The medicine identification portion 8a identifies a medicine. The inhalation volume sensor 12 measures one of the vital capacity and the inhalation volume during breathing at rest of a user. The determination portion 8b determines an optimum inhalation volume for the medicine based on the information. In this case, a difference in the vital capacity and the like due to the physical condition of the user on a day on which treatment is provided can be taken into account.

A well-known measurement unit may be used as the inhalation volume sensor 12 for measuring an inhalation volume. The well-known measurement unit includes a hot wire type and Karman vortex type. The inhalation volume sensor 12 may include a pressure sensor and an arithmetic operation portion. In this case, the arithmetic operation portion obtains the volume of air inhaled per unit time at each point in time based on information transmitted from the pressure sensor, and integrates the volumes over time, so as to obtain the inhalation volume.

### Second Embodiment

FIG. 3 illustrates a second embodiment. Here, a memory portion 13 is attached to the apparatus in FIG. 2. The inhalation volume sensor 12 for measuring an inhalation volume measures the vital capacity and the inhalation volume during breathing at rest of a user. The vital capacity and the inhalation volume during breathing at rest may be registered in the memory portion 13 in advance before the determination portion 8b determines the optimum inhalation volume for the medicine. In this case, for a user who has a small change in the vital capacity and the inhalation volume at rest, it is not necessary to measure the vital capacity and the like every time inhalation treatment is performed.

### Third Embodiment

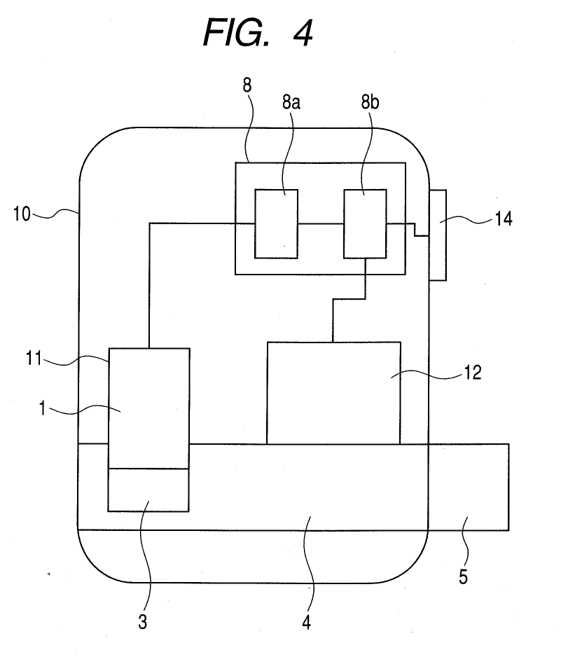

FIG. 4 illustrates a third embodiment. Here, a display portion 14 as an informing unit is provided. The display portion 14 displays the optimum inhalation volume determined by the determination portion 8b to inform a user of an appropriate inhalation volume before the user starts inhalation. The user learns the optimum inhalation volume before inhalation, and can be mentally prepared for the inhalation. Accordingly, the user can more reliably inhale the optimum inhalation volume.

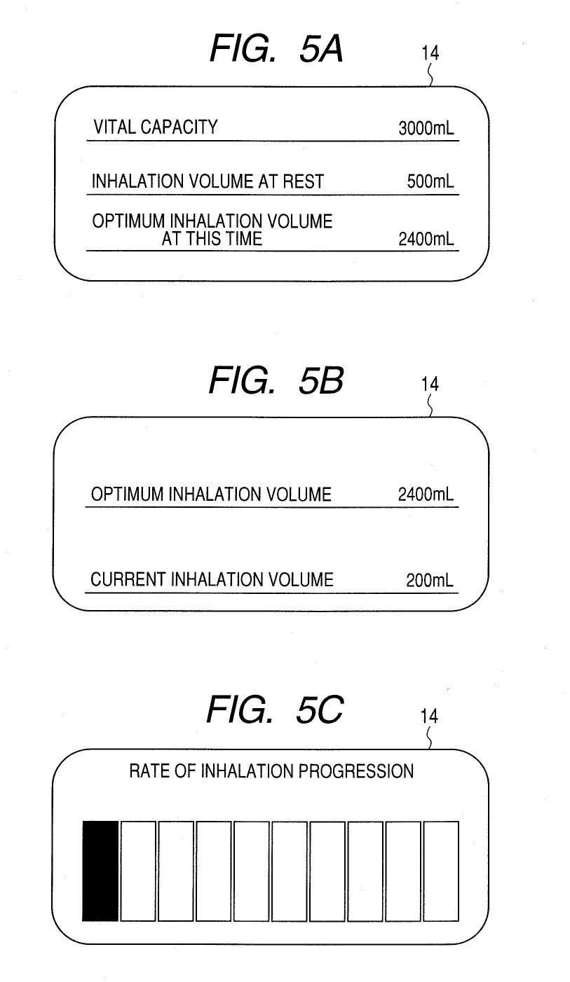

FIGS. 5A, 5B and 5C are views illustrating a content displayed on the display portion 14 at the time of informing a user of the optimum inhalation volume before inhalation. FIG. 5A displays a comparison between the vital capacity and the inhalation volume at rest of a user measured by the inhalation volume sensor 12, and the optimum inhalation volume determined by the determination portion 8b. In this case, the user can learn the comparison between the vital capacity and the inhalation volume at rest of himself or herself and the optimum inhalation volume displayed on the display portion 14. The user can thereby more accurately set a target for the optimum inhalation volume.

The unit of informing a user of the optimum inhalation volume before inhalation is not limited to the display portion 14 as a display unit, and may be an informing unit using sound.

The display portion 14 may also inform a user of information indicating a relation between the inhalation volume measured by the inhalation volume sensor 12 and the optimum inhalation volume determined by the determination portion 8b during inhalation. FIG. 5B is a view illustrating another example of the displayed content. In this case, the optimum inhalation volume based on information transmitted from the determination portion 8b and an air inhalation volume inhaled by the user by the point in time based on information transmitted from the inhalation volume sensor 12 are displayed as numeric values. The user can thereby learn the remaining amount of air to inhale. The user is less likely to stop inhalation before reaching the optimum inhalation volume. Accordingly, the user can more reliably inhale the optimum inhalation volume.

When a user uses the inhaler of the present invention, medicine ejection needs to be completed before the air inhalation volume reaches the optimum inhalation volume.

During inhalation, a user is informed of the information indicating the relation between the inhalation volume measured by the inhalation volume sensor 12 and the optimum inhalation volume determined by the determination portion 8b. In this case, the display portion 14 can be arranged at a position where the user can easily check the displayed content during inhalation. In FIG. 4, the display portion 14 is arranged on the same surface of the inhaler as the suction port 5 as one example of the arrangement. The user can easily check the displayed content of the display portion 14 even during inhalation.

The unit of informing a user of the information indicating the relation between the inhalation volume measured by the inhalation volume sensor 12 and the optimum inhalation volume determined by the determination portion 8b during inhalation is not limited to the display portion 14 as a display unit. For example, the unit includes two light emitting units such as LEDs. One of the light emitting units emits light having a light intensity corresponding to the optimum inhalation volume and the other of the light emitting units emits light having a light intensity corresponding to the current inhalation volume. Such light emitting units can also inform a user of the relation between the inhalation volume measured by the inhalation volume sensor 12 and the optimum inhalation volume.

FIG. 5C is a view illustrating still another example of the displayed content of the display portion 14. In this case, information about the percentage of the air inhalation volume inhaled by a user by the point in time in relation to the optimum inhalation volume according to each medicine is provided to the user during inhalation through a display. The number of blacked out rectangles shows the percentage of the current inhalation volume in relation to the optimum inhalation volume. The user can naturally learn the remaining inhalation volume to reach the optimum inhalation volume from the displayed content during inhalation. The user is less likely to fail in inhalation by stopping the inhalation in midstream before reaching the optimum inhalation volume. Accordingly, the user can more reliably inhale the optimum inhalation volume.

Also, when the inhalation volume measured by the inhalation volume sensor 12 has reached the appropriate inhalation volume, the display portion 14 may inform a user that the inhalation volume has reached the appropriate inhalation volume. For example, a message such as “inhalation has been completed” or “END” is displayed. The user can thereby finish inhalation at the point in time. In the case, the user is less likely to inhale more air than the appropriate air inhalation volume. Accordingly, the user can more reliably inhale the optimum inhalation volume.

The unit of informing a user that the inhalation volume measured by the inhalation volume sensor 12 has reached the appropriate inhalation volume is not limited to the display portion 14 as a display unit. For example, the unit may include a light emitting unit such as an LED, a sound unit such as a speaker, and a vibration unit such as a motor.

### Fourth Embodiment

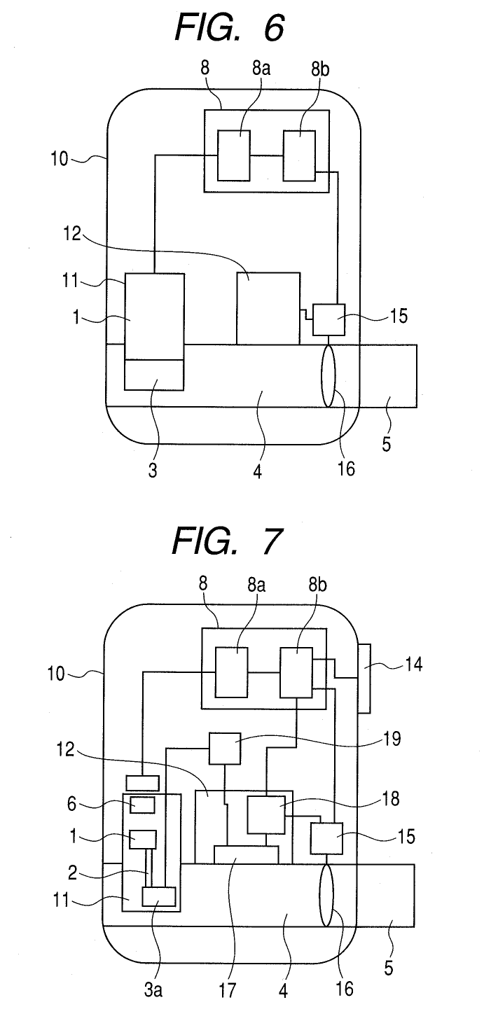

FIG. 6 illustrates a fourth embodiment. Here, an electromagnetic valve 16 as a prohibition unit for prohibiting inhalation by a user is arranged in the air flow path 4. The electromagnetic valve 16 is driven by an electromagnetic valve drive portion 15 connected to the inhalation volume sensor 12. The electromagnetic valve 16 is in an open state before a user starts inhalation. When the air inhalation volume inhaled by the user by the point in time has reached the optimum inhalation volume based on the information transmitted from the inhalation volume sensor 12, the electromagnetic valve 16 is closed by the electromagnetic valve drive portion 15. The air flow path 4 is thereby closed. The user cannot inhale air any more. As described above, the air flow path 4 is closed when the inhalation volume has reached the optimum inhalation volume. Thus, the user cannot inhale more air than the appropriate inhalation volume. Accordingly, the user can more reliably inhale the optimum inhalation volume.

A well-known prohibition unit for closing the air flow path 4 may be used as the prohibition unit for prohibiting inhalation by a user. For example, a shutter may be used instead of the electromagnetic valve.

The prohibition unit for prohibiting inhalation by a user can be located in the air flow path 4 on the suction port 5 side from the medicine ejection portion 3. When the prohibition unit is located as described above, a medicine does not leak out from the medicine ejection portion 3 even when a negative pressure is generated in the closed air flow path 4 by a user's continuing effort to inhale air (in reality, the user cannot inhale air).

### Example

FIG. 7 illustrates an inhaler according to one example. In the present example, a thermal jet head 3a of the thermal jet system is used as a medicine ejection unit. The thermal jet head 3a ejects a medicine by applying thermal energy to the medicine by using the electrothermal transducer. The thermal jet head 3a is disposed in contact with the air flow path 4 so as to eject a medicine into the air flow path 4. Other configurations are the same as those in the apparatus shown in FIG. 6.

A pressure sensor 17 is disposed in contact with the air flow path 4. The pressure sensor 17 measures a negative pressure in the air flow path 4. An arithmetic operation portion 18 performs an arithmetic operation based on the negative pressure detected by the pressure sensor 17. The inhalation volume sensor 12 can thereby measure the inhalation volume of a user during inhalation, and the vital capacity and the inhalation volume at rest of the user. When a certain negative pressure is generated, a head drive portion 19 outputs a signal. The thermal jet head 3a ejects a medicine into the air flow path 4 based on the signal. The user inhales the medicine through the suction port 5.

Before the user starts inhalation, the electromagnetic valve 16 is in an open state. When the air inhalation volume inhaled by the user by the point in time based on the information transmitted from the arithmetic operation portion 18 has reached the optimum inhalation volume, the electromagnetic valve 16 is closed by the electromagnetic valve drive portion 15. The air flow path 4 is thereby closed. Thus, the user cannot inhale air any more.

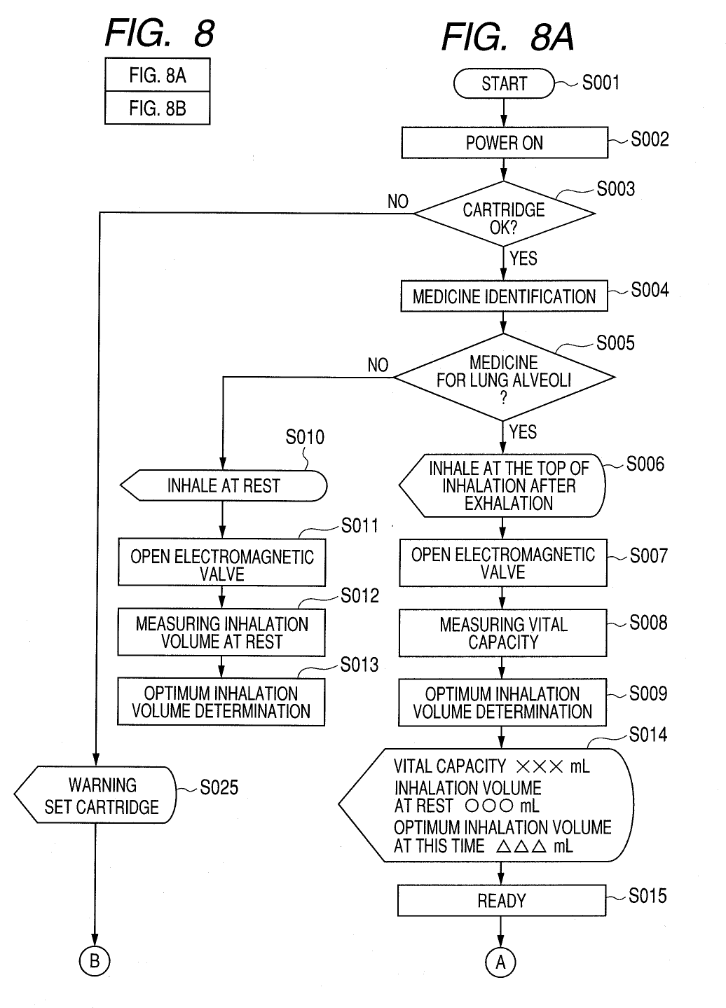

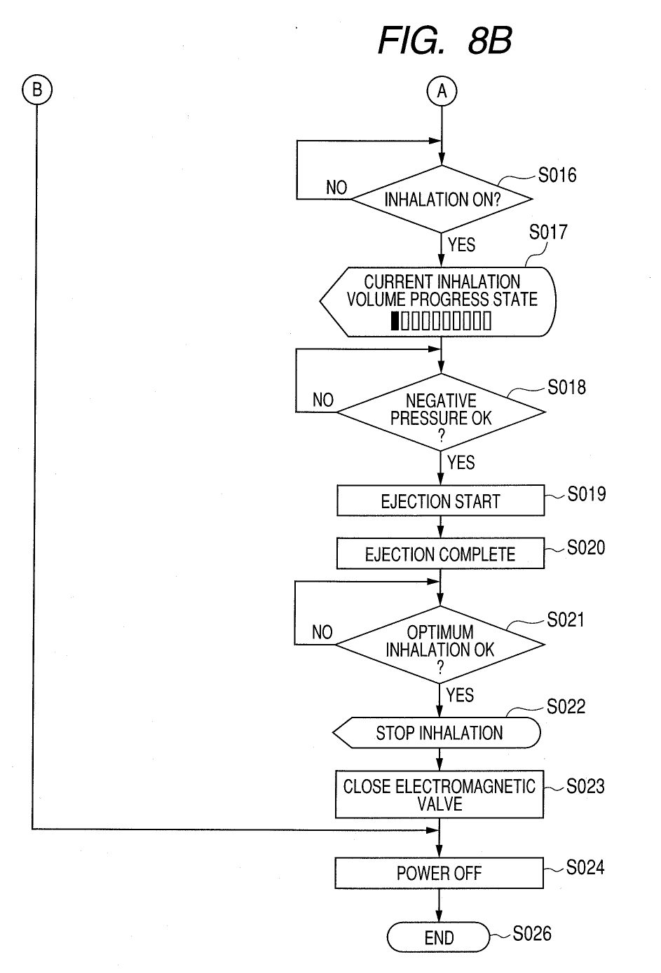

A usage example of the inhaler according to the present example will be described based on the flowchart shown in FIGS. 8A and 8B.

First, a user presses a power switch of the inhaler body to start using the inhaler (step S001). The inhaler is thereby powered ON (step S002). A reading portion (CCD) 9 of the inhaler reads the authentication code 6 attached to the cartridge 11, to check whether the cartridge 11 is set or not (step S003). If the cartridge 11 is not set, the display portion 14 displays a message to prompt the user to set the cartridge (step S025). The inhaler is powered OFF (step S024). The process is completed (step S026).

The medicine identification portion 8a can identify the type of medicine stored in the medicine storing portion 1 of the cartridge 11 based on information regarding the type of medicine transmitted from the reading portion 9 (step S004).

When the medicine is insulin (the medicine which targets lung alveoli), the display portion 14 displays a message to prompt the user to measure the vital capacity (step S006). The electromagnetic valve 16 is opened (step S007). The inhalation volume sensor 12 measures the vital capacity (step S008). In the inhalation volume sensor 12, the arithmetic operation portion 18 obtains the volume of air inhaled per unit time at each point in time based on the information transmitted from the pressure sensor 17, and integrates the volumes over time, so as to obtain the vital capacity. The determination portion 8b determines a volume of 80% of the vital capacity of the user transmitted from the arithmetic operation portion 18 as the optimum inhalation volume (step S009).

When the medicine is salbutamol (the medicine which targets a bronchial region), the display portion 14 displays a message to prompt the user to measure the breathing volume at rest (step S010). The electromagnetic valve 16 is opened (step S011). The inhalation volume at rest is measured (step S012). The inhalation volume at rest can be measured using the inhalation volume sensor 12 in the same method as described above. The determination portion 8b determines a volume equal to the inhalation volume at rest of the user transmitted from the arithmetic operation portion 18 as the optimum inhalation volume (step S013).

After the optimum inhalation volume is determined, the display portion 14 displays the vital capacity and the inhalation volume at rest of the user, and the optimum inhalation volume of this time (step S014). Accordingly, the user is given a target for the optimum air volume which the user will inhale. The inhaler then waits for an inhalation start (step S015).

When the user starts inhalation (step S016), the display portion 14 displays the percentage of the current inhalation volume in relation to the optimum inhalation volume (step S017).

When the inhalation speed of the user is increased, a certain negative pressure or higher is generated in the air flow path 4 (step S018). At this point, the head drive portion 19 transmits a signal instructing medicine ejection based on the information from the pressure sensor 17. The medicine is ejected from the thermal jet head 3a (step S019). The ejection is completed after a given period of time (step S020).

When the inhalation volume of the user calculated by the arithmetic operation portion 18 has reached the inhalation volume determined by the determination portion 8b (step S021), the display portion 14 displays a message to prompt the user to stop inhalation (step S022). The electromagnetic valve 16 is closed (step S023), and the user is forced to stop inhalation. Thereafter, the inhaler is powered OFF (step S024). The process is thereby completed (step S026).

The inhaler according to the present invention may be also used for inhalation of medicines other than the medicines for disease treatment.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese

Patent Application No. 2008-121102, filed May 7, 2008, which is hereby incorporated by reference herein in its entirety.

1. An inhaler comprising:
a medicine storing portion for storing a medicine;
a medicine ejection portion for ejecting the medicine;
a suction portion for allowing a user to inhale air including the ejected medicine;
a medicine identification unit for identifying a type of medicine stored in the medicine storing portion; and
a determination unit for determining an inhalation volume to be inhaled by a user according to the type of medicine identified by the medicine identification unit.

2. The inhaler according to claim 1, further comprising an inhalation volume sensor for measuring an inhalation volume inhaled by a user.

3. The inhaler according to claim 1, further comprising an informing unit for informing a user of the inhalation volume determined by the determination unit.

4. The inhaler according to claim 3, wherein the informing unit informs a user of information indicating a relation between the inhalation volume measured by the inhalation volume sensor and the inhalation volume determined by the determination unit.

5. The inhaler according to claim 4, wherein the informing unit provides information about a percentage of inhalation in relation to the inhalation volume determined by the determination unit to a user during inhalation.

6. The inhaler according to claim 3, wherein when the inhalation volume measured by the inhalation volume sensor reaches the inhalation volume determined by the determination unit, the informing unit informs a user that the inhalation volume measured by the inhalation volume sensor reaches the inhalation volume determined by the determination unit.

7. The inhaler according to claim 2, further comprising a prohibition unit for prohibiting inhalation by a user when the inhalation volume measured by the inhalation volume sensor reaches the inhalation volume determined by the determination unit.
